(19) United States
(12) Patent Application Publication
(10) Pub. No.: US 2007/0106398 A1
(43) Pub. Date: May 10, 2007

(54) Method for regenerating bone

(76) Inventor: Gregory Gene Steiner, Kapolei, HI (US)

Correspondence Address: GREGORY GENE STEINER, 590 FARRINGTON HWY. #524, SUITE 132, KAPOLEI, HI, 96707, US

(21) Appl. No.: 11/268,186

(22) Filed: Nov. 7, 2005

(51) Int. Cl. A61F 2/28

(52) U.S. Cl. 623/908

(57) ABSTRACT

The present invention relates to the methods of treating periodontal disease and bone resorption in the maxilla or mandible comprising the inversion of the adjacent periosteum. The adjacent periosteum is inverted in an effort place the proper regenerative cells in the area of tissue loss in a mammal in need thereof with the goal of regenerating lost periodontium and alveolar bone.

### FIELD OF THE INVENTION

Tissue regeneration is a common goal in medicine and dentistry. However, unique to dentistry is periodontal disease which results in loss of bone and connective tissue attachment to the teeth. Bone regeneration is used to fill bony defects caused by trauma or disease. However bone regeneration alone is inadequate in treating periodontal disease due to the need to regenerate not only bone but also a fibrous connective tissue attachment to the root in order to support the tooth and prevent invagination of the epithelium. If the diseased tissue is removed without an effort to rebuild the lost connective tissue attachment and bone the therapy will fail due to the redevelopment of an epithelial lined pocket that will eventually become reinfected.

Barriers are used when regenerating lost periodontal tissue in order to exclude the epithelium and hopefully permit the regeneration of the periodontal ligament and lost bone. This procedure is commonly referred to as guided tissue regeneration. However, these inert barriers do not contribute vital cells and can result in delayed healing, infection and rejection. Also the barriers do not prevent migration of graft material but only prevent in growth of undesirable tissue around the collar of the tooth. To date the most successful treatment for regenerating lost periodontal tissue is the combination of an inert barrier membrane that covers a bone graft material. The barrier membrane prevents the invasion of epithelium while the bone graft material serves as a filler to maintain space and hopefully contribute to an increased amount of bone growth. The best results that have been achievable with current technology is regeneration on average of between 2 and 3 mm of lost periodontal support. This invention outlines a novel method of regenerating lost periodontal tissue by using vital regenerative tissue to cover the bone graft material while at the same time preventing migration of the bone graft material. Covering the graft material with vital tissue that contains fibrogenic, cementogenic and osteogenic cells attached to the recipient graft site is a novel procedure resulting in increased regeneration without the cost and complications of barriers used in guided tissue regenerative surgery.

### BACKGROUND OF THE INVENTION

Bone regeneration has been a goal of bone graft surgery since the early 1900's. Many bone graft materials have been designed in an effort to restore the skeleton to normal form and function. Recently bone graft surgery has been attempted to reshape bone in an effort to improve esthetics. Many bone graft surgeries use autografts taking bone from one part of the body to be used in another part of the same patient. Other bone graft surgeries use allografts using bone from another member of the same species most often harvested from cadavers. Zenografts are composed of tissue from another species such as coral or cow bone. Recent years have seen the development of synthetic bone graft materials most of which are composed of calcium phosphate based compounds designed to fill the bony defect. Synthetic bone grafts are designed to permit the body of the host to grow into the graft site and encompass the synthetic bone graft material or resorb the synthetic bone graft material and replace the bone graft with host bone. The majority of the bone grafts used to fill osseous defects are granular and are carried to the graft site with appropriate carriers. Mechanical barriers membranes have been developed to exclude undesirable tissue from invading and interfering with regeneration of the lost tissue.

However, non vital barriers do not provide the regenerative cells necessary to produce bone, cementum and periodontal ligament. As a result the bone grafts are often resorbed before osteogenic cells are able to arrive in the graft site or the bone graft material is encased in fibrous connective tissue and no bone regeneration occurs. The barriers do not encapsulate the bone graft and hold it in place but merely cover the bone graft which often results in migration of the bone graft material and invasion of soft tissue under the barrier membrane. At best current barrier technology prevents the in growth of undesirable tissue.

Current barrier technology provides inadequate and incomplete results because cells that resorb the graft material arrive at the site of the bone graft much quicker that cells that build bone. Osteoblasts largely migrate into the bone graft from the surrounding bone. However, osteoclasts and phagocytes arrive at the bone graft material via the blood supply. Because the blood supply will deliver osteoclasts and phagocytes to the bone graft quicker than the osteoblasts can migrate into the graft much of the bone graft is resorbed before osteoblasts arrive. Also, in normal bone osteoblasts direct the osteoclasts regarding which areas need remodeling. When osteoblasts are absent osteoclasts only view the graft material as foreign and proceed to remove the graft material until osteoblasts arrive to direct the action of the osteoclasts.

The placement of a barrier membrane isolates the periodontal defect from the gingiva. With this technique the reconstruction of the periodontal ligament must occur by migration of fibroblasts from the remaining periodontal ligament at the base of the defect. This is very slow and results in soft connective tissue occupying the defects as the bone graft is resorbed. When the barrier is removed or resorbed the periodontal pocket reforms in a significant portion of the previous periodontal defect. Clinical experience shows that this form of healing results in between 2 and 3 mm regeneration of lost periodontal ligament with occasionally failure to regenerate any reattachment.

The only true reattachment of periodontal ligament is the ability to form periodontal ligament fibers embedded in cementum. The only cells found to be capable of forming cementum are periodontal ligament fibroblasts and periosteal fibroblasts (Groeneveld M C J Dent Res 1994;73:1588-1592). The periosteum covers the majority of the skeleton. It is composed of two layers. The bottom layer of the periosteum that covers bone is made of osteoblasts and osteoblast progenitor cells. The outer layer of the periostium is composed of dense collagen fibers and fibroblasts. The most critical aspect of regenerating lost periodontium is reattachment of collagen fibers to the root surface which are anchored in cementum. The next phase of regenerating lost periodontal attachment is the regeneration of bone. Current barrier technology is limited due to the lack of cells that posses the ability to produce cementum, periodontal ligament and bone.

This invention places cells with the potential to regenerate cementum, periodontal ligament and bone in the periodontal defect. This invention contains the cells capable of regenerating the cementum, periodontal ligament and bone in a vital tissue with an active blood supply. This invention places the cells with the ability to regenerate the lost periodontium in the periodontal lesion and consequentially immediately populates the periodontal defect with the proper cells in the proper order according to the regenerative process. With the presence of the proper regenerative cells in the periodontal defect there is no need to place a barrier to exclude unwanted cells. This invention therefore reduces the cost and complications of using barriers while producing superior regenerative results. This invention retains a vital blood supply to the regenerative cells while encapsulating the bone graft material thereby preventing migration of the bone graft material.

This invention:
provides the periodontal defect with cells capable of regenerating cementum
provides the periodontal defect with cells capable of regenerating periodontal fibers.
provides the periodontal defect with cells capable of regenerating bone.
provides the periodontal defect with cells capable of regenerating cementum, periodontal ligament and bone in the proper sequence.
provides regenerative tissue with a functioning blood supply.
provides the bone graft with a covering that encapsulates the bone graft and prevents migration.
reduces the cost of surgery by not requiring an inert barrier membrane.
avoids the adverse reactions associated with inert barrier membranes.
provides regenerative tissue that does not require harvesting from a donor site.
produces superior regenerative surgical results.

### PRIOR ART

Bone grafts have been used in the treatment of bony defects caused by periodontal disease, tooth extraction, resorption and trauma for decades. However, the success of these bone grafts was limited because of a lack of regenerative cells in the graft site. In bone graft surgery a flap in raised and the graft in placed in the defect and the gingiva is sutured over the graft site. However, the cells that are positioned closest to the root surface are epithelial cells and attached gingiva that does not contain cells with the potential to regenerate periodontium or bone. Without cells with the potential to regenerate the cementum, periodontal ligament and bone the result is invasion of the epithelium or soft connective tissue rather than periodontal ligament or bone. The development of mechanical barriers to prevent invasion of the epithelium (U.S. Pat. No. 5,032,445) into the bony defect improved clinical results but still produced limited clinical improvement due to the lack of regenerative cells to convert the bone graft into periodontal ligament and bone. When barrier membranes are used the first cells that arrive into the graft site are osteoclasts and phagocytes that must remove the graft before regeneration of lost tissue can occur. With current technology resorption of the bone graft is far along before regenerative cells are able to migrate into the graft site. The graft site collapses due to resorption of the bone graft before regeneration occurs. When the bone graft is resorbed and the site collapses only a minimal about of regeneration occurs. Another limitation of the use of barrier membranes is their inability to secure the graft material in the graft site. Barrier membranes lay over the graft site and if secured are only secured around the collar of a tooth (U.S. Pat. Nos. 5,032,445 and 5,297,563). This often results in migration of the graft material out from under the barrier membrane and away from the graft site. Also because the membranes are only fixed at the collar of the tooth soft connective tissue can migrate in under the barrier and further limit the effectiveness of the surgery.

The use of connective tissue grafts to function as a mechanical barrier for the treatment of periodontal defects was published by Lekovic et al in 1991 (J Periodontal 1991;61:775-780) and again in 1998 by Lekovic et al (J Periodontol 1998 September;69(9):1050-5) and Kwan et al (J Periodontol 1998 November;69(11):1203-9). In their publications they used connective tissue grafts obtained for the palate and covered the periodontal defect with donor tissue before suturing over the donor tissue with gingival flaps. This procedure produces results similar to improvements found with barrier membranes but suffer from a number of shortcomings. The surgery requires a second operation to obtain the donor tissue form the palate. The amount of donor tissue is limited to small area of the palate that does not contain significant blood vessels and nerves. Due to a limited amount of donor tissue available only small areas can be treated. The procedure significantly increases the complications and pain resulting from the surgery due the need to surgically open a second site to obtain the donor tissue. Donor tissue lacks blood supply which results in significant death of the osteogenic cells of the periostium adjacent to root surface that lacks blood supply. However most importantly the cells placed adjacent to the tooth lack the potential to regenerate cementum and periodontal ligament fibers. When the periosteum is removed from bone with a portion of the soft connective tissue and is laid on the tooth surface osteoblasts and osteoblast progenitor cells cover the root surface. These cells are not able to produce cementum and periodontal ligament fibers. Only the outer layer of the periosteum or the layer furthest from the bone is able to produce cementum and periodontal ligament fibers. When osteoblasts are applied to the root surface reattachment fails to occur. The current invention is different from the previously listed prior art because the periosteum is inverted to place the proper regenerative cells adjacent to the tooth. The current invention is different to the previously mentioned prior art because the osteogenic cells are away from the root and migrate into the defect only after the cells with the potential to produce cementum and periodontal ligament have populated the root surface. The current inventions is different from the previously mentioned prior art because the regenerative cells in the current invention remain attached thereby retaining its blood supply and the vitality of the cells of the periosteum. The current invention is different from the previously mentioned prior art because the bone graft in enclosed thereby preventing migration of the graft material and invasion of soft tissue cells.

Inert barrier membranes do not enclose the bone graft material. This invention delivers cells with the regenerative potential to regenerate the periodontal ligament and bone. Because the cells with regenerative potential are closest to the defect a mechanical barrier is not required. The regenerative cells maintain an intact blood supply ensuring viability. This invention is fixed in all dimensions around the defect effectively preventing migration any bone graft material and preventing soft tissue invasion. The invention provides the ability to deliver regenerative cells to any or all areas of the mouth without the need to take tissue from a donor site. This invention places the regenerative cells in the ideal position for optimum regeneration of the defect. This invention reduces the cost and complications of surgery by eliminating the need for barrier membranes.

### SUMMARY OF THE INVENTION

This invention is a surgical method of delivering cells with regenerative potential to defects in need of repair. This invention utilizes the patient's periosteum to bring regenerative cells into immediate proximity of the defect while maintaining vital blood supply to those cells. The method of this invention encapsulates the defect thereby preventing migration of bone graft material and filling the defect exclusively with fibroblasts that regenerate the cementum and periodontal ligament and osteoblasts that regenerate bone in the proper sequence. This invention eliminates the need of obtaining donor site tissue or using a mechanical barrier thereby reducing both post operative complications and cost of the surgery. This invention improves the outcome of regenerative periodontal surgery and ridge augmentation surgery.

### BRIEF DESCRIPTION OF THE FIGURES

FIG. 1 is a schematic of a lower bicuspid with normal periodontium, bone and tooth.

FIG. 2 is a schematic of a lower bicuspid with periodontal disease.

FIG. 3 is a schematic of a lower bicuspid with a bone graft in place detailing a full thickness surgical flap design with inversion of the periostium over the bone graft.

FIG. 4 is a schematic of a lower bicuspid with a bone graft in place detailing a split thickness surgical flap design with inversion of the periostium over the bone graft.

### DETAILED DESCRIPTION OF THE INVENTION

This invention is a novel use of the patient's periosteum to supply a periodontal defect with cells capable of regenerating lost tissue. This invention covers the defect with vital regenerative cells and places those cells in immediate proximity to the site requiring regeneration. When using this invention the patient's periosteum is exposed using either a full thickness flap or a split thickness flap. The use of a full thickness flap keeps the periostium attached to the soft tissue. A split thickness flap leaves the periostium attached to the bone.

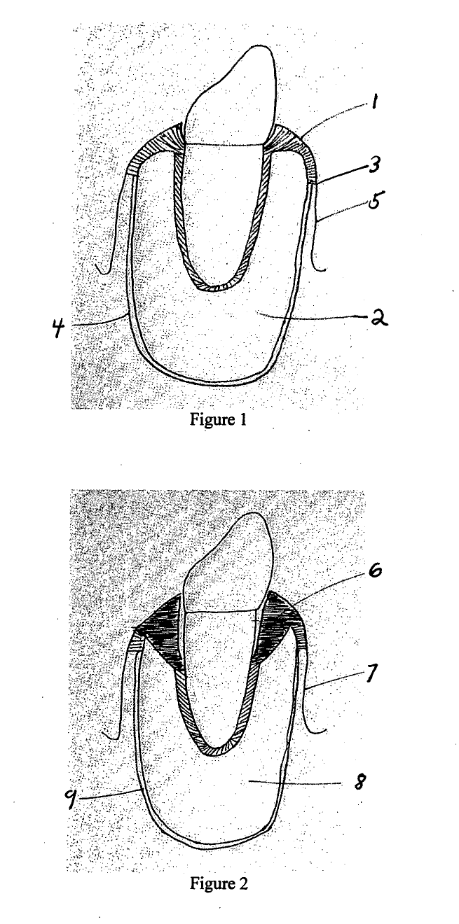

FIG. 1 outlines the normal anatomy of the support structures of a lower bicuspid. Number 1 identifies the attached gingiva which contains collagen fibers that attach the gingiva directly to bone identified as number 2. Number 3 identifies the mucogingival junction which is the point where the attached gingiva stops and the periosteum begins. In this schematic the periosteum (number 4) continues uninterrupted until the keratinized gingiva is encountered on the opposite side of the mandible. Number 5 identifies the mucosa that covers the periosteum.

FIG. 2 is a schematic of the development of periodontal disease around a lower bicuspid. Number 6 identifies the granulation tissue that fills the space once occupied by the periodontal ligament and alveolar bone. Number 7 identifies the mucosa. Number 8 locates the bone of the mandible and member 9 locates the periosteum.

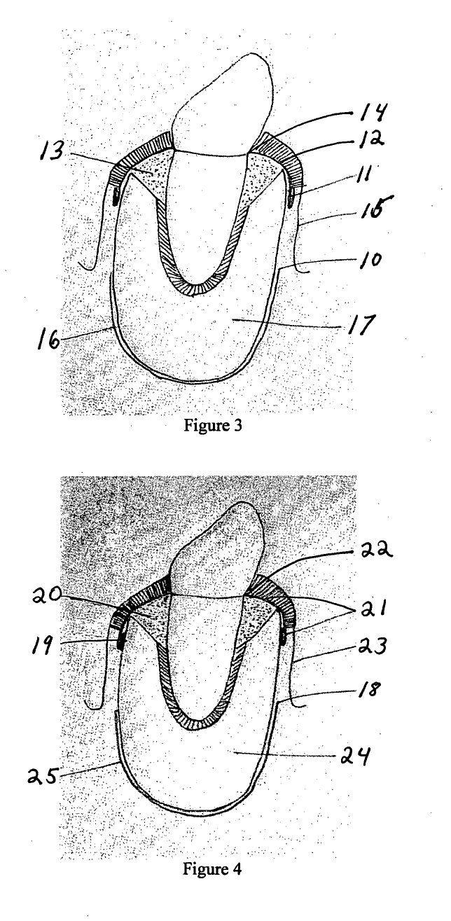

The surgeon will determine before surgery how the periosteum is best acquired. If the surgeon decides to use a full thickness flap the surgeon will leave the periosteum attached to the soft tissue adjacent to the defect site and during the surgery the surgeon will incise the periostium at the base of the flap an adequate distance from defect site. FIG. 3 is a schematic of the full thickness flap approach. Number 10 identifies where the incision is made in the periosteum. After the defect is treated the surgeon will dissect the periosteum off the flap beginning at the line of incision moving coronally. When the periosteum is adequately dissected, the periosteum is inverted over the defect while leaving the periostium attached to the soft tissue adjacent to the defect site. Number 11 identifies where the periosteum remains attached to the gingival flap. Number 12 shows the periosteum that was previously covering bone on the mandible is now inverted and covering the bone graft. Number 13 identifies the bone graft. Number 14 identifies the end of the periosteum which was originally located at number 10. The periosteum originally located at number 10 is sutured adjacent to the tooth in a position determined to best suite the surgical procedure. In this schematic the surgeon has placed the inverted end of the periosteum at the cementoenamel junction. Number 15 identifies the mucosa and number 16 identifies the undisturbed periosteum. In this manner the defect site will be covered with vital inverted periostium which is fixed on the periphery while maintaining blood supply.

If the surgeon decides to use a split thickness flap approach the surgeon will leave the periostium on the bone adjacent to the defect site. FIG. 4 is a schematic showing the use of a split thickness flap design to obtain the periosteum. After a split thickness flap is raised the periosteum is incised at number 18. At the point of incision the periosteum is dissected coronally off the bone. The coronal portion of the periosteum remains attached to the bone as identified by number 19. The defect is often filled with bone graft material as identified by number 20. The periosteum that was elevated from the bone surface is inverted over the defect as identified by number 21. The edge of the periostium that was located at number 18 is now located at number 22. Number 23 identifies the mucosa. Number 24 identifies the bone of the mandible. Number 25 identifies the undisturbed periosteum. In this instance the surgeon has decided to place the edge of the inverted periosteum at the cementoenamel junction of the affected tooth. The inverted periosteum is often fixed in place to maintain contact with the tooth. In this manner the defect site will be covered with vital periostium which is fixed on the periphery of the defect site and sutured around the tooth. In this manner the defect is covered with cells with the ability to regenerate the periodontal ligament and bone. The coronal portion of the periosteum that was not dissected off the bone remains attached thereby encapsulating any bone graft material while maintaining a vital blood supply.

This invention delivers cells to the bony defect and root surface with the ability to regenerate cementum, periodontal ligament and bone. This invention places cells with regenerative potential in immediate proximity to the defect thereby populating the defect with fibroblasts that have the ability to produce cementum, periodontal ligament and osteoblasts that have the ability to produce bone. In normal anatomy the outer layer of the periosteum which is adjacent to the soft connective tissue is comprised of fibroblasts and their progenitor cells. These cells have been found to be able to produce cementum with integrated collagen fibers when placed over dentin (Groeneveld M C J Dent Res 1994;73:1588-1592). In this invention the cells that have the ability to produce cementum and periodontal ligament are inverted and cover the coronal portion of the periodontal defect. These cells are sutured into immediate contact over the defect and in immediate contact with the root surface. In this manner the first cells to populate the periodontal defect are cells of the outer layer of the periosteum which have been shown to posses the ability to produce cementum and periodontal ligament. Because the defect is first populated with the correct cells invagination of the epithelium does not occur and a mechanical barrier is not required.

The most critical phase of regenerative periodontal therapy is reattachment of collagen fibers to the root surface. However regeneration of lost bone needs to follow reattachment of collagen the root surface. The normal anatomy of the periosteum is an outer layer of fibroblasts adjacent to the soft connective tissue with a layer of osteoblasts and their progenitor cells adjacent to bone. The periosteum is thin with the two layers in contact with each other. In this invention the first cells to populate the root surface are the fibroblast and their progenitor cells. By design, with inversion of the periosteum the fibroblasts and their progenitor cells are placed immediately over the periodontal defect with the intent of regenerating cementum and periodontal ligament. However the osteoblasts and their progenitor cells are immediately covering these cells and quickly follow the fibroblast into the defect. During healing the cells with the potential to regenerate cementum and the periodontal ligament first populate the root surface with the osteoblasts and their progenitor cells immediately behind them as they populate the area of bone loss. By design this invention places the proper regenerative cells in the proper location so the proper sequence of formation of cementum, periodontal ligament and bone can occur in order to effect regeneration of the periodontal lesion.

This invention provides a fixed enclosure in all dimensions around and over the defect effectively preventing migration of bone graft material and preventing unwanted soft tissue invasion. This invention provides the ability to deliver regenerative cells to any or all areas of the mouth without the need to take tissue from a donor site. The periosteum is located throughout the mouth starting at the mucogingival junction and covering the maxilla and mandible. As a result an entire arch or entire mouth can be treated at one appointment because it is not limited to the amount of donor tissue available. This invention places regenerative cells in the ideal position for optimum regeneration of the defect. This invention reduces the cost of surgery by eliminating the need for barrier membranes. This invention eliminates many side effects of barrier surgery such as allergic reactions, infection, rejection and the need to remove the foreign object.

In an effort to evaluate the effectiveness of this procedure 10 moderate to severe periodontal lesions were treated using this invention. All test cases resulted in a significant increase in clinical attachment. There was a statically significant difference in gain of clinical attachment in the test group over controls (p<0.5).

This invention is effective for treating defects around teeth and also in the absence of teeth. In the instance of tooth loss the surrounding bone often resorbs resulting in inadequate bone for prosthetic appliances or dental implants. Bone resorption of the maxilla or mandible can also result in esthetic compromises to the teeth and face.

It has been found that cells of the periosteum will produce cementum and periodontal ligament fibers when placed on dentin. However, in the absence of dentin the periosteum does not produce cementum or periodontal ligament. As a result when the periosteum is inverted over a bony defect adjacent to a dental implant only bone is produced. Likewise, in an edentulous ridge when the periosteum is inverted over a bone graft only bone is produced. This invention can be used to rebuild bone lost as a result of tooth loss or trauma. The method of ridge augmentation is the same as used for regeneration of periodontal defects. The same problem exists when attempting to regenerate an atrophied ridge as exists when teeth are present. On the crest of the ridge the attached gingiva attaches to the underlying bone. Attached gingiva has no potential to regenerate bone and in fact appears to inhibit bone regeneration. Any bone graft placed under the attached gingiva lacks the progenitor cells capable of producing bone. This invention solves that problem by inverting the periosteum over the bone graft providing the needed progenitor cells to produce bone in the most critical area of the graft.

A full thickness or split thickness flap is raised and once an adequate amount of periosteum is exposed the periostium is incised at the base of the flap and the periostium is dissected coronally. A bone graft is placed over the ridge and the periostium is sutured over the bone graft. Incisions are made in such a manner so that the suture line closing the periostium over the graft and the suture line closing the gingiva are offset. In this manner the graft material is enclosed and encapsulated in cells with osteogenic potential.

1. A method of regenerating cementum, periodontal ligament and bone by inverting the periosteum apical to a periodontal defect over the periodontal defect in a mammal in need thereof.

2. The method of claim one of inverting the periosteum over a periodontal defect by way of a full thickness flap incising the periosteum on the flap and inverting it over the periodontal defect.

3. The method of claim one of inverting the periosteum over a periodontal defect by way of a split thickness flap incising the periosteum on the bone and inverting it over the periodontal defect.

4. A method of growing bone in the maxilla or mandible by inverting the periosteum over a bone graft in a mammal in need thereof.

5. The method of claim 4 of growing bone in the maxilla or mandible by raising a full thickness flap incising the periosteum on the flap and inverting it over the bone graft.

6. The method of claim 4 of growing bone in the maxilla or mandible by raising a split thickness flap incising the periosteum on the bone and inverting it over the bone graft.

7. The method of claim 4 when the defect is adjacent to a dental implant.

8. A method of evaluating the inverted periosteal surgery for its effectiveness in regenerating lost periodontium by inverting the periosteum over a periodontal defect in a mammal in need thereof.
